(19) United States
(12) Patent Application Publication
(10) Pub. No.: US 2011/0086434 A1
(43) Pub. Date: Apr. 14, 2011

(54) MICROFLUIDIC DEVICE AND METHOD FOR CONTROLLING FLUID FLOW USING THE SAME

(75) Inventor: DONG HO SHIN, DAEJEON (KR)

(73) Assignee: ELECTRONICS AND TELECOMMUNICATIONS RESEARCH INSTITUTE, DAEJEON (KR)

(21) Appl. No.: 12/893,371

(22) Filed: Sep. 29, 2010

(30) Foreign Application Priority Data
Oct. 9, 2009 (KR) 10-2009-0096431
May 31, 2010 (KR) 10-2010-0051082

(51) Int. Cl. G01N 33/53; G01N 30/00; F17D 3/00

(52) U.S. Cl. 436/501; 422/69; 137/1; 137/561 R

(57) ABSTRACT

A microfluidic device includes: a lower plate having a first channel; a first upper plate fixedly stacked on the lower plate and having grooves on an upper portion thereof, a fluid inlet and a fluid outlet formed at positions corresponding to both ends of the first channel; and a second upper plate movably inserted into the grooves of the first upper plate and including a second channel, a hole connected to a right end of the second channel, and a third channel connected to the right side of the hole.

### CROSS-REFERENCE TO RELATED APPLICATIONS

This application claims the priority of Korean Patent Application No. 10-2009-0096431 filed on Oct. 9, 2009, and 10-2010-0051082 filed on May 31, 2010, in the Korean Intellectual Property Office, the disclosures of which are incorporated herein by reference.

### BACKGROUND OF THE INVENTION

1. Field of the Invention

The present invention relates to a microfluidic device and a method for controlling a fluid flow using the same, and more particularly, to a technique for simply and precisely controlling a fluid flow by merely moving a second upper plate in a microfluidic device formed by sequentially stacking (or laminating) a lower plate, a first upper plate fixed to an upper portion of the lower plate, and the second upper plate movably inserted into a groove (or recess) of the first upper plate.

2. Description of the Related Art

The related art technique for controlling a fluid flow includes a technique for controlling the shape and size of a channel, a technique for processing an inner wall of a channel to have hydrophilicity or hydrophobicity, a technique for using pressure or electrical energy, and the like.

The technique for controlling the shape and size of a channel, in which the width and depth of a channel differ to adjust a flow speed of a fluid based on hydromechanics and the shape of the channel is controlled to induce a large or small capillary force, is commonly used to adjust a fluid flow. However, the related art technique for controlling a fluid flow by merely adjusting the shape and size of a channel has a limitation to be applied to a bio-chip field in which capabilities of maintaining of a certain transfer speed of a fluid in a micro-channel, maintaining a certain reaction time in a reactive area, and stopping a fluid transfer, and the like, are requisite for a quantization of analyses.

In addition, the technique for controlling a fluid by treating the inner wall of a channel to have hydrophilicity or hydrophobicity has a limitation to be applied to the bio-chip field in which a fluid is to be stopped at a desired position or transferred to a desired position. For example, there is a technique for stopping a fluid flow by setting a hydrophobic area on a channel in order to maintain a certain reaction time. When a fluid meets the hydrophobic area, the flow of the fluid is stopped due to the qualities of pushing out the fluid of the inner wall of the channel, and in this case, a duration in which the fluid is stopped is proportional to the area and length of the hydrophobic area. In this case, however, in general, although most materials exhibit hydrophobic characteristics at an initial stage, when the duration in which they meet the fluid is lengthened, the materials tend to be changed to have hydrophilicity. Thus, when time goes by, the fluid passes through the hydrophobic area at a very slow speed. When the hydrophobic area of the channel is set to stop the fluid flow to maintain a certain reaction time, the hydrophobicity of the hydrophobic area thus becomes incomplete due to an absorption of ambient moisture, the amount of a reactant, an inertial force of the fluid flow at the reaction area, resulting in a situation in which the reactant at the reaction area may flow out to the hydrophobic area. In addition, if the reaction time is intended to be adjusted with this method, a particular section of the channel must be treated to have hydrophobicity, for which, thus, a suitable material and processing method must be devised in consideration of the physical and chemical characteristics of a fluid used.

In addition, the technique for controlling a fluid flow by using pressure requires a pressure adjusting device such as a syringe pump, a peristaltic pump, and the like, which leads to an increase in the size of a diagnostic system including the system elements, and because the cost for configuring the system is determined by the cost of the pressure adjusting device, rather than by the cost of the system elements, the technique cannot be accepted in a point of care system (POCS) market seeking small and low-cost elements. Also, the technique for controlling a fluid flow by using electrical energy is advantageous in that a system can be reduced in size, but disadvantageous in that it can be applicable only in very limited cases. In addition, in order to apply electrical energy, an electrode must be formed on an element, which requires a very unusual, peculiar form and a scheme according to the characteristics of fluids, and in order to transfer an electrical signal to the interior of the element, various devices must be combined to be configured. Thus, although it is a small, compact system, it is very complicated to fabricate and implement the system. In particular, when one element performs reactions at various stages, if the electrical characteristics of a fluid change by stages, electrical energy must be adjusted by stages, complicating the processes.

### SUMMARY OF THE INVENTION

An aspect of the present invention provides a microfluidic device, fabricated by sequentially stacking a lower plate, a first upper plate fixed to an upper portion of the lower plate, and a second upper plate movably inserted into a groove (or a recess) of the first upper plate, capable of simply and precisely controlling a fluid flow by merely moving the second upper plate.

According to an aspect of the present invention, there is provided a microfluidic device including: a lower plate having a first channel; a first upper plate fixedly stacked on the lower plate and having grooves on an upper portion thereof, a fluid inlet and a fluid outlet formed at positions corresponding to both ends of the first channel; and a second upper plate movably inserted into the grooves of the first upper plate and including a second channel, a hole connected to a right end of the second channel, and a third channel connected to the right side of the hole.

According to another aspect of the present invention, there is provided a method for controlling a fluid flow by using a microfluidic device formed by sequentially stacking a lower plate, a first upper plate, and a second upper plate such that the first upper plate is fixed to an upper portion of the lower plate, and the second upper plate is movably inserted into grooves of the first upper plate, including: injecting a fluid to a first channel formed on the lower plate through a fluid inlet formed on the first upper plate; and when the first channel is filled with the fluid injected through the fluid inlet so the fluid flow is stopped, moving the second upper plate to connect a fluid outlet formed on the first upper plate and a second channel formed on the second upper plate.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is an exploded perspective view of a microfluidic device according to an exemplary embodiment of the present invention;

FIGS. 2a to 2c are perspective views for explaining a method for controlling a fluid flow using a microfluidic device according to an exemplary embodiment of the present invention;

FIG. 3 is a schematic view illustrating a phenomenon in which a droplet is created at a fluid outlet of a first upper plate; and

FIG. 4 is a sectional view showing implementation of an immune reaction by using a microfluidic element according to an exemplary embodiment of the present invention.

### DETAILED DESCRIPTION OF THE PREFERRED EMBODIMENT

Exemplary embodiments of the present invention will now be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. In describing the present invention, if a detailed explanation for a related known function or construction is considered to unnecessarily divert the gist of the present invention, such explanation will be omitted but would be understood by those skilled in the art.

In the drawings, the shapes and dimensions may be exaggerated for clarity, and the same reference numerals will be used throughout to designate the same or like components.

It will be understood that when an element is referred to as being “connected with” another element, it can be directly connected with the other element or intervening elements may also be present. In contrast, when an element is referred to as being “directly connected with” another element, there are no intervening elements present. In addition, unless explicitly described to the contrary, the word “comprise” and variations such as “comprises” or “comprising,” will be understood to imply the inclusion of stated elements but not the exclusion of any other elements.

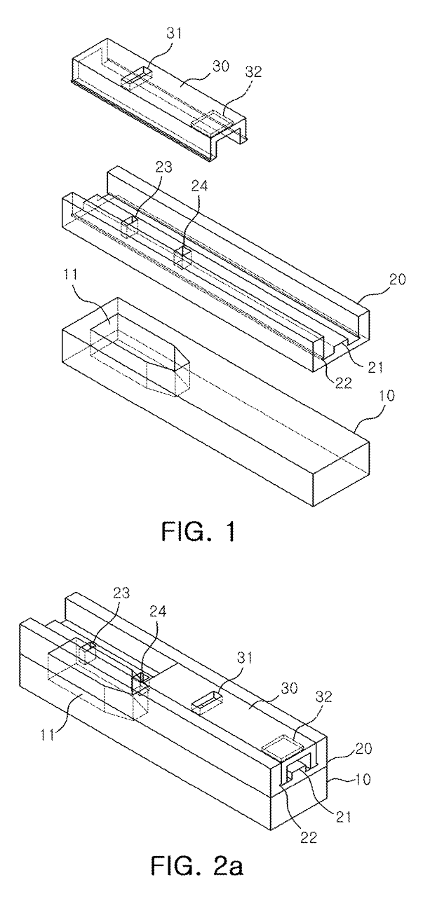

FIG. 1 is an exploded perspective view of a microfluidic device according to an exemplary embodiment of the present invention.

The microfluidic device according to an exemplary embodiment of the present invention includes a lower plate 10 and two upper plates 20 and 30 sequentially stacked on one lower plate 10. In this case, the first upper plate 20 adjacent to the lower plate is fixed on the lower plate 10, and the second upper plate 30 is inserted into grooves (or recesses) of the first upper plate 20 and freely moves along the grooves.

A first channel 11 having a pre-set width and depth is formed at a left portion of the lower plate 10.

The first upper plate 20 includes a groove to allow the second upper plate 30 to be inserted thereinto. A flow path 21 is formed along a central portion of a lower surface of the groove and has a section protruded upward with relation to with the lower surface. Guide rail grooves 22 are formed on a lower portion of the side of the groove, into which side protrusions of the second upper plate 30 are to be inserted. A fluid inlet 23 and a fluid outlet 24 are formed at the flow path 21. The fluid inlet and the fluid outlet 24 are formed at positions corresponding to both ends of the first channel 11 formed on the lower plate 11, respectively.

A channel having a pre-set width and depth is formed on a lower portion of the second upper plate 30 to form a flow path. A hole 31 is formed to penetrate a portion of the second upper plate, and a reservoir 32 is formed to be spaced apart from the hole 31 at the right of the hole 31. As for the channel formed on the second upper plate 30, the portion of the channel at the left side of the hole 31 will be referred to as a second channel, and a between the hole 31 and the reservoir 32 will be referred to as a third channel.

The foregoing lower plate 10, the first upper plate 20, and the second upper plate 30 are sequentially stacked to form a microfluidic device having a two-storied structure. Flow paths along which a fluid flows are formed on the respective layers. The fluid is first injected into a flow path of the lower layer, and as the second plate 30 is moved, the flow path of the lower layer and that of the upper layer are connected to allow the fluid to flow from the lower layer to the upper layer.

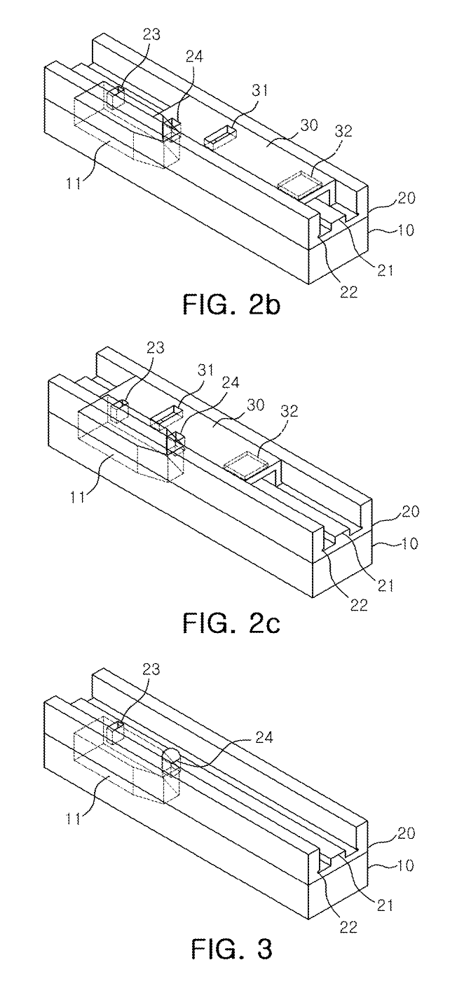

FIGS. 2a to 2c are perspective views for explaining a method for controlling a fluid flow using a microfluidic device according to an exemplary embodiment of the present invention.

First, in the structure in which the lower plate 10, the first upper plate 20, and the second upper plate 30 are sequentially stacked, as shown in FIG. 2a, the movable second plate 30 is positioned at an upper portion of the first upper plate 20 at the rightmost side.

In the state as shown in FIG. 2a, when the fluid is injected through the fluid inlet 23 formed on the first upper plate 20, the fluid flows along the first channel 11 formed on the lower plate 10 and then is stopped at the fluid outlet 24. When a sufficient amount of fluid is introduced into the first channel 1 through the fluid inlet 23, the fluid flows up through the fluid outlet 24 to form a droplet as shown in FIG. 3. In this case, because the upper layer does not have a channel connected with the fluid outlet 24, the droplet is maintained to have a certain size and form unless force is applied to, the fluid inlet 23, whereby the fluid is stopped.

Thereafter, the second upper plate 30 is moved to the left, so that the second channel of the second upper plate 30 can be connected to the fluid outlet 24 as shown in FIG. 2b. Then, the fluid, which has moved along the first channel 11, flows to the second channel of the second plate 30 through the fluid outlet 24 and then moves up to a portion connected with the hole 31 along the second channel. Soon, the fluid meets the hole 31, an empty space, so it cannot proceed any further and is stopped.

Thereafter, the second upper plate 30 is moved to the left, so that the third channel of the second upper plate 30 can be connected to the fluid outlet 24 as shown In FIG. 2c. Then, the fluid remaining in the interior of the first channel 11 flows through the third channel, a new flow path, to reach the reservoir 32.

In this manner, in the microfluidic device according to an exemplary embodiment of the present invention, the movement and stopping of the fluid can be accurately controlled by simply moving the second upper plate.

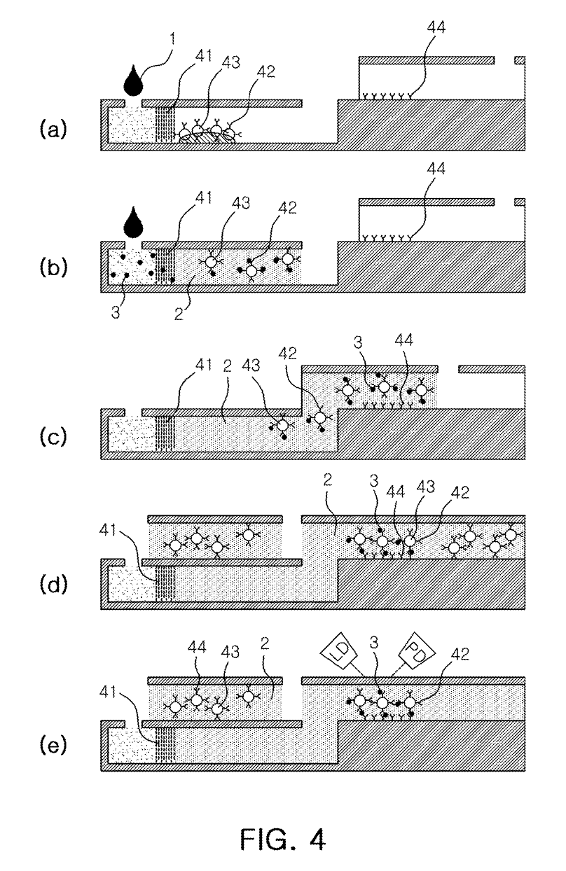

FIG. 4 is a sectional view showing the implementation of an immune reaction by using a microfluidic element according to an exemplary embodiment of the present invention. Specifically, FIG. 4 illustrates an example of implementing reactions of several steps such as a sandwich immunoassay in one element.

As shown in FIG. 4(a), in a state in which the second upper plate is positioned at the rightmost side, when blood 1 is injected through the fluid inlet, blood corpuscles included in the blood are removed by a filter 41 installed in the interior of the first channel and only blood plasma (serum) 3 passes through the filter 41 to reach an area in which first-stage antigen-antibody reaction of sandwich immunoassay occurs.

A fluorescent nanoparticle-detection antibody assembly formed of a fluorescent substance or fluorescent nanoparticles 43 and detection antibodies 42 are physically or chemically bonded is coated and formed at a first-stage antigen-antibody reaction area, namely, a portion at the right side of the filter 41, and a reaction portion reacting with a particular material is positioned.

As shown in FIG. 4(b), when the blood plasma component 3 fills the first-stage antigen-antibody reaction area entirely, the fluid flow is stopped and a sufficient reaction takes place. At this time, the blood plasma component 3 meets the fluorescent nanoparticle-detection antibody assembly coated on the first-stage antigen-antibody reaction area, and because the fluid is stopped and cannot proceed any further, until such time as the second upper plate is connected to the fluid outlet, the antigen-antibody reaction can take place with a sufficient time.

Thereafter, as shown in FIG. 4(c), when the second upper plate is moved to the left so the second channel of the second upper plate is connected to the fluid outlet, the fluorescent nanoparticle-detection antibody-blood plasma assembly moves to the second channel of the upper layer through the fluid outlet. A specific antibody 44, which is specific to an antibody, is fixedly formed at an entrance of the second channel, and a detection unit is positioned to detect the fluorescent nanoparticle-detection antibody-blood plasma assembly. Thus, the fluorescent nanoparticle-detection antibody-blood plasma assembly, which has moved to the second channel, meets the specific antibody 44 of the detection unit included in the second channel to undergo a second-stage reaction. In this case, the fluid, filling up the second channel, cannot proceed any further by the open hole and is stopped.

When the second-stage reaction is sufficiently made, as shown in FIG. 4(d), the second upper plate is moved to the left, so that the third channel of the second upper plate can be connected to the fluid outlet. Accordingly, the fluorescent nanoparticle-detection antibody-blood plasma assemblies, which have not been bonded with the specific antibody 44 of the detection unit, are mostly shoved to the left side and only the assemblies bonded with the detection unit remain. As the blood plasma components, which have not been reacted with the fluorescent nanoparticle-detection antibody assembly remaining in the first channel, continue to be introduced into the third channel, the assemblies remaining on the surface, rather than having been reacted, shoved to the left side to reach the reservoir. Accordingly, the non-specifically reacting elements, without having participated in the reaction, are all removed.

Thereafter, as shown in FIG. 4(e), the amount of the fluorescent nanoparticles bonded with antibodies is measured by using LD/PD.

Thus, as described above, the microfluidic device can accurately control the movement and stopping of the fluid through the simple operation of moving the second upper plate, and allow unnecessary fluids not participating in the reaction to flow out and allow a new fluid to be continuously introduced, thus completing the reaction.

As set forth above, according to exemplary embodiments of the invention, in the microfluidic device formed by sequentially stacking the lower plate, the first upper plate fixed to the upper portion of the lower plate, and the second upper plate movably inserted into a groove of the first upper plate, a fluid flow can be simply and precisely controlled by moving the second upper plate.

Also, because the microfluidic device has a simpler structure, elements can be implemented to be compact and small and at a low cost, and the microfluidic device can be extensively utilized regardless of the types of fluids in use.

In addition, an analysis that performs reactions of several stages, like, a sandwich immunoassay, can be implemented by the microfluidic device, and a fluid can be more accurately analyzed by precisely controlling a fluid flow.

While the present invention has been shown and described in connection with the exemplary embodiments, it will be apparent to those skilled in the art that modifications and variations can be made without departing from the spirit and scope of the invention as defined by the appended claims.

What is claimed is:

1. A microfluidic device comprising:
a lower plate having a first channel;
a first upper plate fixedly stacked on the lower plate and having grooves on an upper portion thereof, a fluid inlet and a fluid outlet formed at positions corresponding to both ends of the first channel; and
a second upper plate movably inserted into the grooves of the first upper plate and including a second channel, a hole connected to a right end of the second channel, and a third channel connected to the right side of the hole.

2. The device of claim 1, wherein the first upper plate further comprises a flow path having a section protruded upward compared with a lower surface of the grooves along the lower surface of the grooves and guide rail grooves allowing side protrusions of the second upper plate to be insertedly positioned therein.

3. The device of claim 1, wherein the second upper plate further comprises a reservoir connected to the right end of the third channel.

4. The device of claim 1, wherein the first to third channels have a pre-set width and depth, respectively.

5. The device of claim 1, wherein when the second upper plate is positioned at the rightmost side of the first upper plate and the fluid injected to the first channel through the fluid inlet fully fills the first channel, the fluid flow is stopped.

6. The device of claim 5, wherein when the second upper plate moves to the left so the fluid outlet and the second channel are connected, the fluid, which has filled the first channel, flows to the second channel through the fluid outlet.

7. The device of claim 6, wherein the fluid, which has moved to the second channel, flows along the second channel, and then, when the fluid meets the hole, the fluid is stopped.

8. The device of claim 7, wherein when the second upper plate moves to the left so the fluid outlet and the third channel are connected, the fluid remaining in the interior of the first channel moves to the third channel through the fluid outlet.

9. The device of claim 1, wherein the first channel comprise:
a filter; and
a reaction part formed by coating a fluorescent nanoparticle-detection antibody assembly, and reacting to a particular material which has passed through the filter.

10. The device of claim 9, wherein a specific antibody is fixedly formed at the second channel, and the second channel comprises a detection unit detecting an assembly formed as the fluorescent nanoparticle-detection antibody assembly and the specific material are reacted.

11. A method for controlling a fluid flow by using a microfluidic device formed by sequentially stacking a lower plate, a first upper plate, and a second upper plate such that the first upper plate is fixed to an upper portion of the lower plate, and the second upper plate is movably inserted into grooves of the first upper plate, the method comprising:
injecting a fluid to a first channel formed on the lower plate through a fluid inlet formed on the first upper plate; and
when the first channel is filled with the fluid injected through the fluid inlet so the fluid flow is stopped, moving the second upper plate to connect a fluid outlet formed on the first upper plate and a second channel formed on the second upper plate.

12. The method of claim 11, further comprising:
when the fluid, which has moved from the first channel to the second channel through the fluid outlet, flows along the second channel and is stopped by an empty space, moving the second upper plate to connect the fluid outlet and a third channel formed on the second upper plate.

13. The method of claim 12, further comprising:
performing an antigen-antibody reaction between a reaction portion provided in the interior of the first channel and a target material included in the fluid, after the fluid is injected into the first channel.

14. The method of claim 13, further comprising:
detecting an assembly according to the antigen-antibody reaction by a detection unit provided in the interior of the second channel, after the second channel is connected.

15. The method of claim 14, wherein a non-specific reactive element which has not been detected in the detecting of the assembly is removed by the connecting of the third channel.
